(19) United States
(12) Patent Application Publication
(10) Pub. No.: US 2003/0051262 A1
(43) Pub. Date: Mar. 13, 2003

(54) Animal models for neurodegenerative disease

(76) Inventors: Susan Adele Greenfield, Oxford (GB); John Nicholas Pepys Rawlins, Oxford (GB); Robert Michael John Deacon, Oxford (GB)

Correspondence Address: Nixon & Vanderhye, 8th Floor, 1100 North Glebe Road, Arlington, VA, 22201-4714, US

(21) Appl. No.: 10/169,343

(22) PCT Filed: Dec. 22, 2000
(86) PCT No.: PCT/GB00/04991

(30) Foreign Application Priority Data
Dec. 30, 1999 (GB) 9930825.6

(51) Int. Cl. A01K 67/027

(52) U.S. Cl. 800/9; 800/12; 800/18

(57) ABSTRACT

The present invention provides animal models for neurodegenerative diseases, in particular, for example, Alzheimer's Disease, which rely on introduction into the brain of a biologically active fragment of acetylcholinesterase of sequence AEFHRWSSYMVHWK (SEQ. ID no.1), or an active variant thereof, to produce brain lesions.

This invention relates to animal models for neurodegenerative disorders, in particular Alzheimer's disease, and to methods for providing them. In particular, the invention relates to the use of a peptide fragment from close to the C-terminus of acetylcholine esterase (AChE) for inducing cognition impairment so as to provide an animal model for neurodegenerative disorders.

Alzheimer's disease (AD) is a degenerative brain disease, the incidence of which rapidly increases with advancing age. Certain populations of brain cells progressively die, particularly but by no means exclusively those using acetylcholine as a neurotransmitter. Recently modern imaging techniques have revealed how the medial temporal lobe area, which contains the hippocampus (a vital structure for learning and memory generally in humans and for certain types of spatial learning in animals) progressively shrinks as Alzheimer's disease runs its course.

A sizeable minority of cases of Alzheimer's disease appear to have a genetic component (familial Alzheimer's) but the majority are sporadic occurrences with no known precipitating factors, although there are positive correlations with previous brain damage, low intelligence, and possibly aluminium concentration in drinking water. Cigarette smoking and folic acid appear to lower the incidence.

The principle symptoms of Alzheimer's disease are steadily progressive loss of cognitive faculties such as memory (particularly recent episodic memories), problems with language and speech such as difficulty in finding the right words, and attention. Multi-infarct dementia, the most common other form of dementia, often presents a similar clinical picture but as it is due to a series of small strokes its progression is more stepwise. Other clinically problematic associated symptoms of Alzheimer's are depression, aggression and eventually incontinence. Moderate to advanced cases of Alzheimer's require 24 hour care, and thus the disease, which can affect 20% or more people over 80 years old., is enormously costly.

A number of proposals have previously been made for providing an animal model for Alzheimer's Disease. However, none of these have proved entirely satisfactory.

By way of example, transgenic mice have been produced which are genetically modified to produce larger than normal amounts of β-amyloid in the brain on the basis that this protein is found in plaques associated with development of AD. Some such mice show learning and memory deficits but have been criticised as acceptable models for AD since plaques can occur in normal ageing and the concentrations of β-amyloid do not necessarily correlate with the degree of dementia.

Other recent attempts to provide a good animal model for AD have been premised on the hypothesis that depletion of cholinergic neurons in relevant areas of the brain is of paramount importance. Post-mortem investigations of brains of AD patients have demonstrated that the cholinergic projection from the nucleus basalis of Meynert (NBM; also known as the nucleus basalis magnocellularis) to areas of the cerebral cortex is the pathway that is most early and severely affected in AD patients. Hence, a number of studies have looked at stereotaxic injection of neurotoxins into the NBM of rats to produce reduction in cortical cholinergic activity. A serious limitation of such studies accounting for unreliable behavioural results has previously been suggested to be lack of selectivity of the neurotoxin for cholinergic cells. To overcome this limitation, BigI and Schliebs more recently proposed selective lesion of basal forebrain cholinergic neurons in rats by injection into the NBM of a cytotoxin (saporin) coupled to a monoclonal antibody to the nerve growth factor receptor associated with such neurons (BigI,V. and Schliebs, R. (1998) Simulation of cortical deficits—a novel experimental approach to study pathogenic aspects of Alzheimer's disease, J. Neural. Transm. [Suppl] 54: 237-247). This approach to modelling AD is, however, also now considered open to question as a reliable approach as the behavioural deficits observed can be inconsistent or insubstantial. Indeed, the above-noted paper of BigI and Schlieb itself reports unsatisfactory behavioural results obtained by others using the same agent to cause cholinergic lesions in rat brains.

Significantly, although depletion of acetylcholine is prominent in AD, it is not the only characteristic. Another reason for disillusion with cholinergic models of AD has been poor clinical results from cholinergic therapy. Although tacrine, donepezil and rivastigmine, all AChE inhibitors, are the only currently licensed therapies for AD, this is primarily due to lack of more effective alternatives rather than their therapeutic efficacy, which is generally considered to be very limited. Also, toxicity and accompanying side effects can limit the usefulness of current therapies.

It has now been shown that cognitive impairment, e.g. attentional deficit, in rats reminiscent of that characteristic of AD patients can be produced by injection into the NBM and other brain areas of a 14 mer fragment of AChE having the sequence AEFHRWSSYMVHWK (SEQ. ID. no. 1) or a biotinylated version of that peptide. Explanation for the effectiveness of this approach for mimicking AD can be founded on previous in vitro evidence implicating non-enzymatic action of AChE in the etiology of a number of neurological disorders including AD. However, the present specification for the first time presents evidence showing that a fragment of AChE alone can produce in vivo cellular degeneration and thereby neurological dysfunction reminiscent of neurological dysfunction associated with a known neurological disease. Importantly, this approach to modelling AD, in direct contradiction to the proposal of BigI and Schliebs, cannot be attributed to a selective cholinergic deficit.

AChE is an enzyme whose classical or cholinergic role is to degrade extracellular acetylcholine. However, it has long been known that AChE can be found associated with non-cholinergic neurons. Consistent with this, in recent years there has been growing evidence that AChE has a non-enzymatic role, although the biochemical basis for this function remains to be fully elucidated.

Published International Application WO 97135962 presents preliminary evidence indicating that the peptide of SEQ. ID no. 1 (referred to hereinafter for simplicity as AChE peptide) is capable of modulating induced Ca²⁺ flux into neurons, e.g. neurons of the substantia nigra in slices of guinea pig midbrain. It has been postulated that an in vivo counterpart of the peptide of SEQ. ID. no. 1 is responsible for mediating non-enzymatic function of AChE in the brain. It has been hypothesised that such non-enzymatic action of AChE underlies trophic function in developing brains but if activated in adult brains leads to neurodegenerative disorders. Based on knowledge of AChE neuronal location and in vitro studies with the peptide of SEQ. ID. No. 1, this hypothesis of disease causation is currently of particular interest in relation to Alzheimer's Disease, Parkinson's Disease and Motor Neuron Disease (Greenfield, Spring research News (1997) 2-3; Greenfield, Brit. Med. J. (1998) 317, 19-26). However, as previously indicated above, direct evidence that the AChE peptide alone will produce neurodegenerative disease has been lacking.

WO 97/35692 does refer to infusion of a low dose of the AchE peptide via a cannula into one substantia nigra of a rat followed by systemic administration of amphetamine. Such-treatment did give rise to behavioural disturbance, but such rats do not represent a useable model for neurodegeneration since they show increased neuronal activity rather than decrease of neuronal activity associated with cell death. More particularly, such animals do not provide any guidance for establishing a useful animal model for Alzheimer's Disease.

Significantly, AChE is present in the neuritic plaques and neurofibrillary tangles found in the cortex of Alzheimer brains (Carson et al. Brain. Res. (1991) 540, 204-208). However, interest in such plaques, as indicated above, has previously focussed on the β-amyloid component. Interestingly, as shown in FIG. 1 of WO 97/35962, and in FIG. 1 of the present specification, SEQ ID no. 1 (representing amino acid residues 535 to 548 of mature AChE) is conserved between AChE of different species, including human and rat AChE, and exhibits similarity to the N-terminal region of β-amyloid peptide 1-42.

It has now been found that surprising histological changes occur when the AChE peptide is injected into the brains of rats. These are unlike anything normally seen with conventional neurochemical lesion. In particular, a lesion rapidly forms when the peptide is introduced into a region of the brain which is linked to the hippocampus by nerve projections, specifically the septal nuclei and/or the diagonal band of Broca (S/DB) region of the brain. This region provides major projections to the hippocampus (via the bundle of nerve fibres known as the fornix) and cingulate cortex. The lesion is an order of magnitude larger than that caused by a conventional neurotoxin such as NMDA, and the rapidity with which it forms is also remarkable (less than an hour; time course studies using MRI (magnetic resonance imaging) of the brain can illustrate this). Striking behavioural changes result which can be easily assessed by using standard tests of hippocampal function which are widely considered to be models of the functions affected in Alzheimer's disease. The physical changes, which include significant cell damage at and around the site of introduction of the peptide, can be observed by histological studies.

There is also provided herein for the first time evidence that attentional deficit reminiscent of that observed in Alzheimer's patients can be effectively modelled by injection of biotinylated AchE peptide (SEQ. Id no. 1) into the NBM of rats (see Example 4). Such deficit in cognitive function is associated with more subtle lesions than the AChE peptide lesions referred to above and cannot be correlated with loss of cortical cholinergic loss as made evident by comparison with comparable-injections of NMDA. Importantly, comparable injection of NMDA was found to give a greater reduction in the level of cortical choline acetyltransferase (a measure of cholinergic loss) but without a selective effect on performance in the set attentional task (a serial choice reaction task as further discussed below).

### SUMMARY OF THE INVENTION

The invention therefore provides in one aspect a method of providing an animal model for a neurodegenerative disease which comprises introducing, e.g. injecting, an effective amount of a peptide having the sequence:

AEFHRWSSYMVHWK (SEQ ID. no. 1)

or an active variant of the peptide, into one or more sites in the brain of a non-human animal whereby said peptide causes cellular degeneration and thereby impairment of a testable brain function, wherein impairment of the same brain function in a human is indicative of a neurological disorder. It is predicted that such a method is applicable to modelling any neurological disorder associated with non-enzymatic function of AChE, in-particular, for example, Alzheimer's Disease, Parkinson's Disease and Motor Neuron Disease. However, there is especial interest in adoption of this approach for modelling Alzheimer's Disease in which case the testable brain function of interest will be a cognitive function, e.g. attentional deficit. It will be appreciated that a method of the invention as described above above may further comprise testing for impairment of an appropriate brain function, e.g. by providing the animal with an attentional task to test for attentional impairment.

Prior to, simultaneously or after the peptide, a test agent may be administered. In this case, the animal model will be subsequently tested for the brain function of interest, e.g. attention, to determine whether the test agent inhibits, prevents or increases impairment of the relevant brain function. Of particular interest are compounds thus identified which will inhibit or prevent impairment of brain function associated with administration of the peptide alone and which can be formulated for passage across the blood-brain barrier.

The invention will be further described below with reference to the figures detailed below.

### BRIEF DESCRIPTION OF THE FIGURES

In the attached figures:

FIG. 1 shows a multiple sequence alignment of five AChE sequences, three BuChE sequences and the human amyloid precursor protein (Hum Amyl) at the region of interest. Hum AchE=human AchE; Rab AchE=rabbit AchE; Mus AchE=mouse AchE; Bov AchE=bovine AchE; Hum BuCHE=human butyrylcholinesterase (BuChE); Rab BuChE=rabbit BuChE; Mus BuChE=mouse BuChE. Residues in bold are conserved across all sequences. Boxed residues are shared by all AchEs and human amyloid precursor protein but by none of the BuChEs. The β-amyloid peptide 1-42 is shown by the boxed area. The bar above the alignment shows the position of the AchE peptide (SEQ. ID. No. 1). The bar below the alignment shows the position of the homologous peptide fragment at the N-terminus of β-amyloid 1-42.

FIG. 2 is a graph showing the weight of rats (mean +/−SEM) on days 1 to 10 following injection with 2 μl 33 mM AchE peptide (also known as Peptide B), 2 μl 33 mM NMDA or 2 μl water into the medial septum/vertical limb of the diagonal band of Broca (S/DB).

FIG. 3 shows the results of T-maze tests pre-operatively and two weeks post-operatively for the same rats as described in Example 2. Veh=controls injected with water; NMDA=rats injected with NMDA; B=rats injected with AchE peptide.

FIG. 4 shows the results of 20 massed T-maze trials as referred to in Example 2.

FIG. 5 shows T-maze results for the same rats when a 45s delay was imposed between sample and choice trials as discussed in Example 2.

FIG. 6 shows the locomotor activity for the rats referred to in Example 2 as tested on 2 successive days for 4 hours in standard locomotor activity cages following completion of the T-maze testing.

FIG. 7 shows comparison of the weights of the rats referred to in Example 2 pre-operatively and I month post-operatively prior to microscopical brain examination.

FIG. 8 shows the arrangement of the septal nuclei and their connections with the hippocampus in diagrammatic form.

FIG. 9 shows area of tissue loss in the medial septal region of rats anaesthetised and injected as described in Example 2 with AchE peptide (B), the equivalent peptide from BuChE (C), a scrambled version of AchE (ScrB), NMDA or water (W) (*p less than 0.05 vs water).

FIG. 10 is a schematic representation of some ascending cholinergic pathways in the rat brain. Abbreviations: h, hippocampus; ms, medial septal nucleus; nb, nucleus basalis; nc, neocortex; nv1, nh1, nuclei of diagonal band, lie adjacent to medial septum.

FIG. 11 shows a rat testing apparatus for performing a serial choice reaction task test for attention as described by Higgs et al., European J. Neuroscience (2000) 12, 1781-1788.

FIG. 12 shows the result of decreasing light stimulus duration on ability of non-treated rats to perform an attentional task in apparatus as shown in FIG. 11. The right panel shows the % of correct lever-light trials. The left panel shows the number of incorrect choices which were errors to the centre tray (which would have been correct had no light been presented).

FIG. 13 is a line drawing of a coronal section of the right side of a rat brain to show the site of microinjection of AchE peptide as described in Example 4. The microinjection site within the NBM is shown as a dark spot at the base of the internal capsule and globus pallidus. Abbreviations: AT=anterior thalamus; C=cortex, CC=corpus callosum; CP=corpus striatum; F-fimbria-fomix; GP=globus pallidus; IC=internal capsule; Rt=reticular nucleus of the thalamus; VL=lateral ventricle; V3=third ventricle.

FIG. 14 shows the results of testing rats in apparatus as shown in FIG. 11 following injection into the NBM of 2 μl water, 2 μl 16.5 mM biotinylated AChE peptide-(referred to in FIG. 14 as Synaptica Peptide) or 2 μl 16.5 mM NMDA as described in Example 4. Results are also shown for a fourth group of hippocampal lesioned rats. The left panel shows the proportion of correct trials as a proportion of the total trials, The right panel more specifically indicates where rats went on incorrect trials. Each column represents correct trials/correct trials plus incorrect trials to centre. The right panel thus shows the tendency for each group to go to the centre tray as if no light had been. presented (rather than respond wrongly to the wrong side).

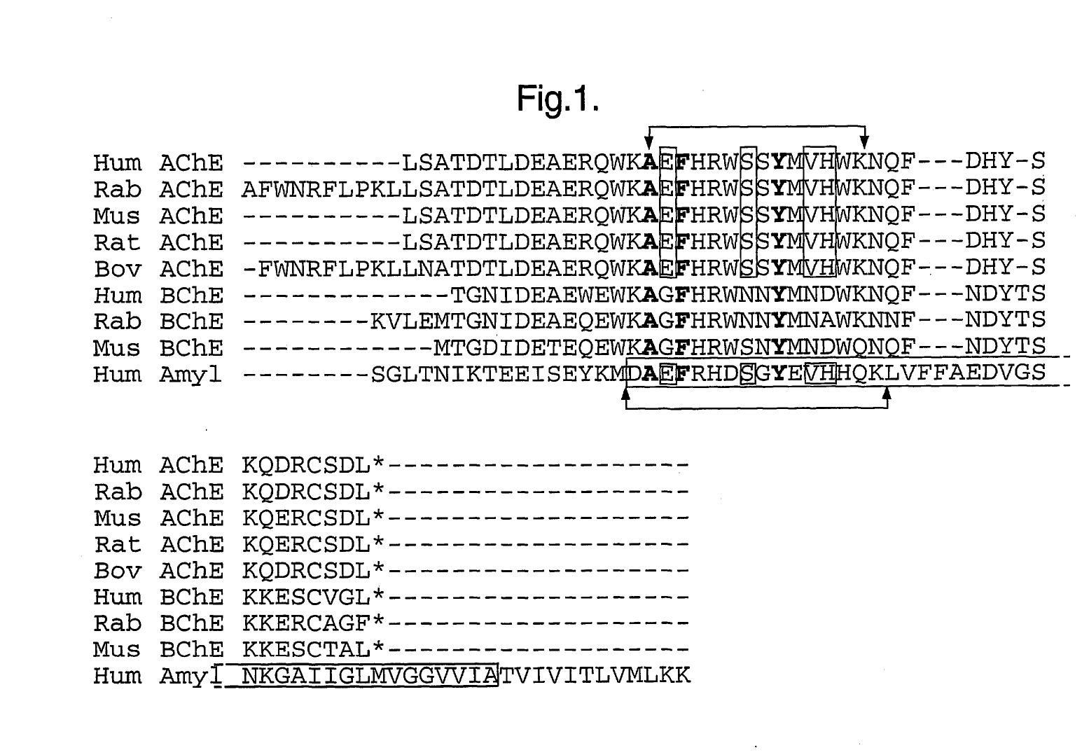

### DETAILED DESCRIPTION

Of particular interest, for example, so far as an animal model for Alzheimer's disease is concerned is the introduction of the peptide into a site in the septohippocampal cholinergic system, preferably the S/DB region. A single injection here of a sufficient dose can have pronounced effects on behaviour. Alternatively or additionally, the peptide may, for example, be introduced into a site in the cortical cholinergic system, preferably in the nucleus basalis/substantia innominata.

As indicated above, an especially preferred embodiment comprises introducing AChE peptide or an active variant thereof, e.g. especially the biotinylated form of SEQ. ID. No. 1 or an active variant thereof, into the nucleus basalis magnocellularis region (or nucleus basalis of Meynert) of a non-human animal, preferably a rodent, so as to produce lesions which can be linked to attentional impairment. An appropriate concentration range for the peptide may be established by conventional histological methods for identifying lesions in animal brains and/or by recognised behavioural tests. A concentration of biotinylated AChE peptide as high as 16.5 mM has been employed satisfactorily in preliminary studies but more physiological doses may prove preferable,

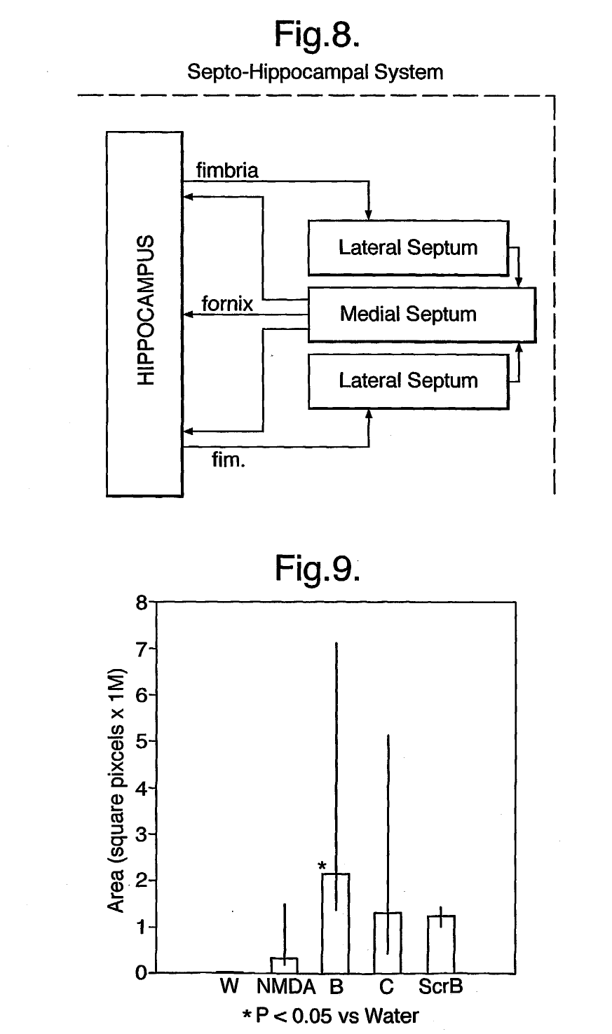

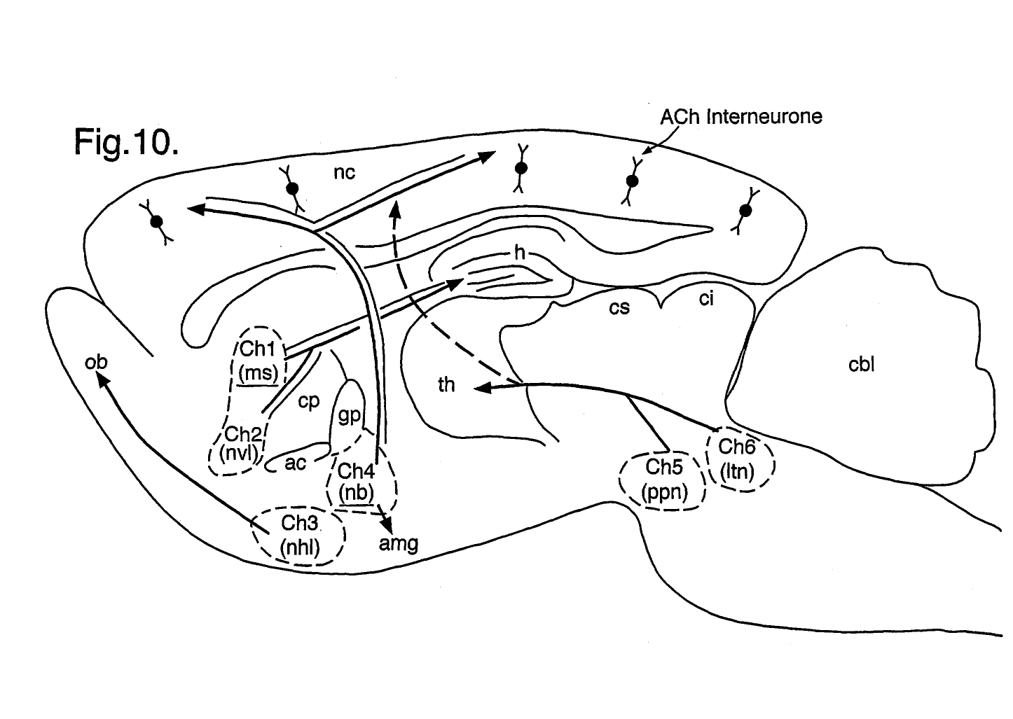

The septohippocampal cholinergic system is a communication system which operates between the S/DB and the hippocampus, along connecting neurones (see FIG. 8) The cortical cholinergic system is a separate cholinergic system. As previously indicated above, it starts in the nucleus basalis region of the brain. Neurons within the NBM send long projections to most areas of the cerebral cortex, including the prefrontal and parietal regions, which are involved in attention. In rats, the nucleus basalis/substantia innominata regions overlap and are ill-defined. The term nucleus basalis magnocellulularis or nucleus basalis of Meynert (NBM) as used herein will therefore be understood to equate with the whole area emcompassing the NBM which provides projections to the cortex.

Both the cortical and septohippocampal cholinergic systems contribute to memory. Memory impairment in rats with lesions of the nucleus basalis/substantia innominata and medial septum including cell bodies of the cortical and septohippocampal cholinergic systems, respectively, have been compared previously (Miyamoto et al. Brain Research (1987) 419:,19-31). Memory impairment was observed in both cases, although this was far less marked in the case of lesions in the cortical cholinergic system. Nevertheless, as previously indicated above, the NBM is now viewed by the inventors in this instance as a preferred site at which the AChE peptide, or an active variant thereof, may be introduced to produce behavioural changes that represent behaviour in Alzheimer's disease.

A method of the invention for preparing an animal model of Alzheimer's Disease may include testing for one or more aspects of cognitive function known to be affected in Alzheimer's Disease. Peptide treated animals may thus be tested for one or more of impairment of memory, learning, attention and problem-solving. Particularly suitable for testing animals such as rats are working spatial memory tests, such as the T maze test described herein. Other standard tests which can be applied include for example the Morris water maze and the radial arm maze.

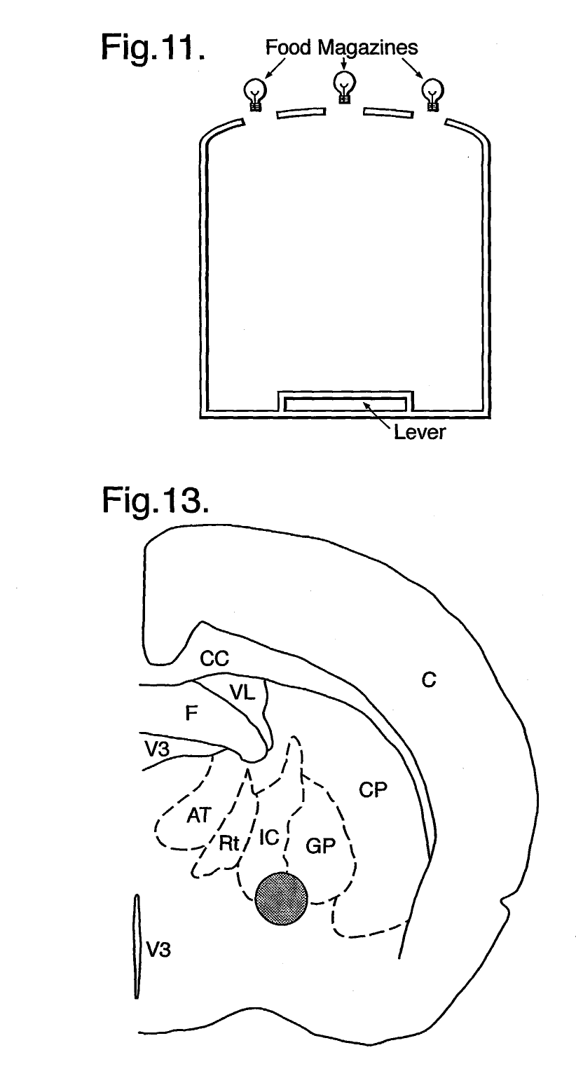

It is particularly preferred to combine production of peptide lesions in the NBM with testing for attentional deficit using apparatus providing a serial choice reaction task. Rats can be trained to perform simple attentional tasks, such as to push open a panel with their nose when a light flashes behind it and retrieve a food reward. Although sensitive to treatments affecting attention, a failure to respond in such a test might also be due to an effect on performance. The treatment might for example cause sedation. A serial choice reaction task addresses this concern by providing for more than one stimulating event, for example lever-pressing by a rat may result in one of three events: a light flash from a left magazine or a right magazine or no light in which case the correct choice is a central magazine. Suitable apparatus for testing for attentional impairment in this manner has been described in Higgs et al., European J. Neuroscience (2000) 12, 1781-1788 (see also FIG. 11).

Other behavioural functions which can be monitored include but are not limited to social behaviour, emotional reactivity, contextual conditioning, pre-pulse inhibition of startle reflex, two-way aversive conditioning and motivation as measured by food and water intake or sucrose preference.

As indicated above, the peptide employed in preparing an animal model in accordance with the invention may be the AchE peptide (SEQ. ID. No. 1) itself or an active variant thereof, including modified forms of that peptide having modified amino acid residues, e.g. the biotinylated form. Variants of the AchE peptide include peptides having one or two or a few amino acid substitutions and/or one or two or a few amino acid deletions and/or a one or two or a few additional amino acid residues. compared to SEQ. ID. No. 1. A suitable variant may for example have an N-terminal and/or C-terminal extension. It may be the in vivo counterpart of the peptide of SEQ. ID. no. 1.

Given SEQ. ID. No. 1 as a guide for comparison, it is a straightforward matter to make variant peptides and test them for efficacy in a method according to the invention. For example, one might start by testing a peptide which is identical to the AChE peptide except for one or two conservatively substituted amino acid residues. Conservative substitutions can be predicted on the basis of amino acid properties which are well characterised. Active variants of the AchE peptide for use in accordance with the invention may also possibly be determined by in vitro tests of peptides for retention of calcium channel modulatory activity. For this purpose, guinea pig midbrain slices may, for example, be employed for electrophysiological studies as described previously in Published International Application no. WO 97/35962. Alternatively, for example, organotypic tissue culture of hippocampal slices, e.g. from rats, may be used.

Suitable variants of the AChE peptide for use in the invention are expected to be peptides containing at least six amino acid residues and having at least 70% sequence identity with part or all of the AChE sequence above. Preferably, peptides for use in the invention are expected to contain at least 12 amino acid residues and have at least 90% sequence identity with SEQ. ID. No. 1.

As indicated above, subtle lesions in the NBM of rats giving rise to attentional deficit have been found to be achievable by use of biotinylated AchE peptide. However, it is envisaged that functionally equivalent lesions in the NBM may be achieved by use of other peptides as discussed above.

The source of the peptides described herein for use in the invention is not material to the invention. They may be for example synthetic peptides prepared by chemical synthesis, or they may be prepared from larger peptide or polypeptide molecules by enzymatic digestion, or they may be produced by recombinant techniques.

The chosen peptide will normally be administered by stereotaxic injection into an anaesthetised animal, although administration to conscious animals through implanted cannulae may sometimes be preferred, e.g. to examine acute effects (30 minutes duration) without anaesthesia. Alternatively, pressure microinjection or electrophoresis through a (glass) micropipette may be preferable for ionophoresis recordings.

In another aspect, the invention provides non-human mammals, e.g. rodents, treated according to a method described herein. It will be appreciated from the discussion above that in a particularly preferred embodiment such animals are known to exhibit impaired cognitive function, e.g. attentional deficit, as a result of a lesion in the brain, in particular the destruction of cells in or associated with the septohippocampal or cortical cholinergic system, especially for example destruction of cells in the NBM as a result of injection therein of biotinylated AchE peptide or an effective variant thereof. Although the examples below, relate to studies with rats, it will be appreciated that the invention is not confined to such animals but also extends to other animals which may be treated in accordance with the invention to produce neurological dysfunction, including, for example monkeys.

As indicated above, particularly favoured are animals according to the invention which represent models for Alzheimer's disease or at least an aspect of that disease. For instance, such models can be used to study potential cognition enhancing agents, or to test generally for agents having biological activity relating to neurodegeneration.

In addition to animal models for Alzheimer's disease, the invention also envisages, however, animal models for a range of neurodegenerative disorders, including but not limited to Parkinson's disease, motor neuron disease, and prion-related disorders such as bovine spongiform encephalopathy and Creutzfeldt-Jakob disease (CJD, including “new variant” CJD). For example, a rat injected with the AChE peptide described herein in the substantia nigra region of the brain may be useful as a model for Parkinson's disease and thus for testing reagents to assess their potential as therapeutic agents for treatment of Parkinson's disease.

As previously indicated above, a method of the invention may further comprise administering prior to, simultaneously, or after the peptide a test agent and determining whether said agent can inhibit, prevent or increase impairment of the testable brain function of interest, e.g. in the case of an animal model for Alzheimer's Disease, attention as determined preferably by, for example, performance of a serial choice reaction task, and/or can inhibit, prevent or increase cellular damage in the brain. The test agent may be a compound administered in any conventional manner for a therapeutic agent whereby it can gain entry to the brain. Alternatively, it may be a cellular transplant introduced into the brain.

In a further aspect, there is provided a method of testing an agent for biological activity in a neurodegenerative disease, which method comprises administering the agent to an animal model as described herein and assessing the animal for any change (improvement or deterioration) associated with the brain lesion. Such assessment will comprise determining whether said agent will inhibit, prevent or increase impairment of an appropriate testable brain function, e.g. a cognitive function such as attention or memory, and/or determining whether there is any improvement of deterioration in cellular damage at the relevant site(s) in the brain.

A test agent identified as above which inhibits or prevents impairment of a testable brain function constitutes a further aspect of the invention. A pharmaceutical composition comprising such a test agent together with a pharmaceutically acceptable carrier or diluent also forms part of the invention.

A method of assessing a test agent as described above may further comprise synthesising a selected compound found to inhibit or prevent impairment of a testable brain function. Such synthesis may be followed by incorporation of the synthesised compound into a pharmaceutical composition. It will be appreciated that of particular interest are such compounds which can be formulated to pass through the blood-brain barrier. Such compounds may be of therapeutic use in treating a neurological disorder such as Alzheimer's Disease.

In a further aspect, there is thus provided use of an agent selected in accordance with the invention as above which inhibits or prevents impairment of an appropriate testable brain function of the animal model for the manufacture of a medicament for use in the treatment of a neurological disorder. In a preferred embodiment, such use is use of an agent which has been shown to inhibit or prevent impairment of a cognitive function , e.g. attention, associated with injection of an appropriate peptide into the brain of a rat to model Alzheimer's Disease.

In more detail, the findings on which this invention is based are as follows.

Initial injection of the AChE peptide into the dorsal hippocampus of rats was found to cause cell loss in a limited area around the site of injection and cortical damage around the injection tract. The neuronal loss in the hippocampus itself, however, appeared less than from injections of the known “reference” neurotoxin N-methyl-D-aspartate (NMDA) in equivalent concentrations.

The comparatively small amount of histological damage after intrahippocampal AChE peptide injection suggested that in order to see any behavioural effects from AChE peptide given intracerebrally it would be necessary to inject it at multiple sites within the hippocampus. To produce a complete lesion of the hippocampus in a rat using the reference neurotoxin NMDA, 14 injections are required on each side of the hippocampus, making 28 in total. As noted above, the damage from AChE peptide was more localised than that from NMDA, so even more injections would probably be necessary. This would be technically difficult.

It was then discovered that by injecting the peptide into a different site, at the border of the medial septum and the diagonal band of Broca (S/DB) region of the brain, a much larger lesion could be produced which also affected the hippocampus via the fornix connecting system, even with only a single injection. Furthermore, the lesion could be identified using simple behavioural tests known to be affected by hippocampal dysfunction.

The medial septum is a small compact structure that has a powerful influence on other parts of the brain, and hence on behaviour. It projects nerve terminals (axons) into large regions of the hippocampus. The medial septum lies next to the vertical limb of the diagonal band of Broca (VDB), which innervates the cingulate cortex above the hippocampus and may play a role in some aspects of learning, memory, attention and emotional behaviour.

These results were unexpected as there was a major problem anticipated with this approach. Selective chemical lesions of the septum normally produce much smaller effects than more traditional techniques involving mechanical or electrolytic destruction of brain tissue. These much smaller effects require very much more sophisticated tests to assess them, involving extensive animal training and complex apparatus, impractical for a workable animal model. This, however, proved not to be the case here.

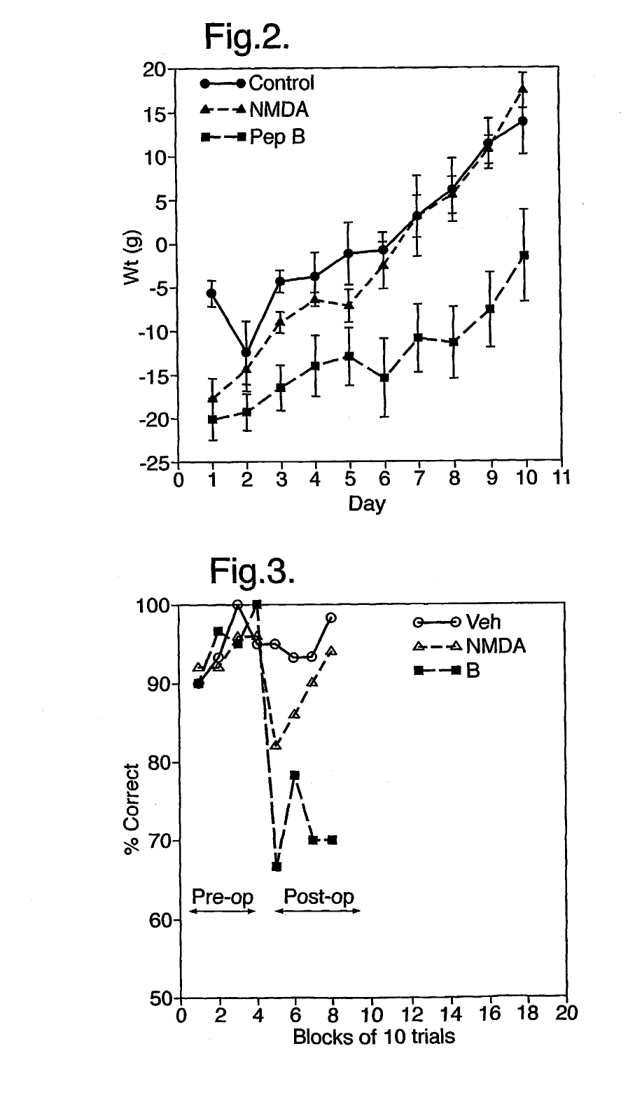

Rats injected with AChE peptide in the S/DB showed a greater weight loss than controls after surgery, before recovering to near control weights. NMDA-treated rats showed a similar initial weight loss to AChE peptide-treated rats, but a much faster recovery over subsequent days (see FIG. 2).

When tested for working spatial memory on a T-maze (on which all groups had been trained prior to operation, when they showed near-perfect performance) control rats continued their excellent performance, NMDA-treated rats showed an initial dip in performance but soon recovered to control levels, whereas AChE peptide-treated rats showed a larger drop than the NMDA-treated rats. Unlike NMDA-treated rats, the AChE peptide-treated group's performance did not recover significantly (see FIGS. 3, 4 and 5). Two rats (out of the six AChE peptide treated rats) were at virtually chance levels throughout testing, a characteristic sign of a large lesion in the hippocampal system.

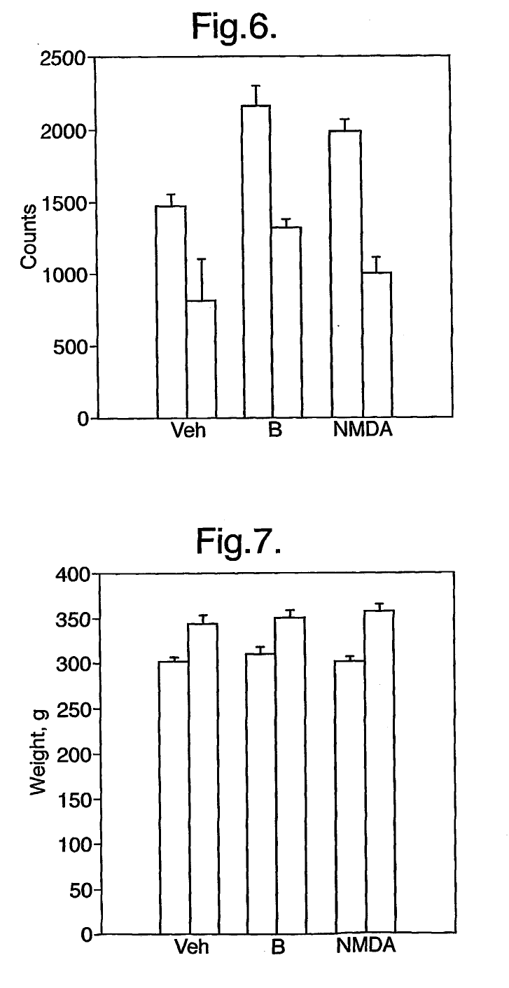

When tested for locomotor activity, the AChE peptide treated rats were significantly hyperactive compared to controls (another sign of hippocampal dysfunction). The two rats which were worst on the T-maze were the most active in the locomotion test. NMDA-treated rats were slightly more active than controls but this was not statistically significant (see FIG. 6).

At post-mortem, the two AChE peptide treated rats which were worst on the T maze and most hyperactive showed gross atrophy of the septum (see FIG. 9) and of the fornix, the band of nerve fibres which carries projections from the medial septum to the hippocampus.

These experiments indicated that the AChE peptide can cause considerable central nervous system damage, which appears to be an order of magnitude greater than that of the benchmark neurotoxin NMDA. Cytotoxins normally only affect cell bodies; the gross damage to the fibre tracts of the fornix suggests a different or additional mode of action of AChE peptide. Studies suggest that the S/DB area of the brain may be particularly susceptible to damage. These striking results were unexpected and could not have been predicted from the effects of AChE peptide on tissue cultures which are disclosed in published International Application WO 97/35962.

Neurotoxic effects have also been observed by injection of the AchE peptide into mice. The peptide is expected to act similarly in other non-human animals.

Example 4 below details further studies which have shown that attentional deficit reminiscent of Alzheimer's Disease can be produced by a single injection of biotinylated AchE peptide into the NBM of rats. As indicated above, such rats, combined with testing for attention by means of a serial reaction choice task, are now envisaged as a favoured means of testing agents for potential therapeutic utility in relation to Alzheimer's Disease.

Details for performing a serial choice reaction task for assessment of attention have previously been given above with reference to Higgs et al. European Journal of Neuroscience (2000) 12, 1781-1788 and FIG. 11. The following is a list of some other behavioural tests which will be suitable for use in accordance with the invention. Most but not all of these are tests of cognitive function. Tests which relate to behaviour but not cognitive faculties are also included and may be used instead of or in addition to the tests of cognitive function such as memory.

Attention

Carli, M., Robbins, T. W., Evenden, J. L. and Everitt, B. J. (1983) Effects of lesions to ascending noradrenergic neurones on performance of a 5-choice serial reaction time task in rats; implications for theories of dorsal noradrenergic bundle function based on selective attention and arousal. Behavioural Brain Research 9, 361-380.

Social Behaviour

Gardner, C. R. and Guy, A. P. (1984) A social interaction model of anxiety sensitive to acutely administered benzodiazepines. Drug Dev. Res. 4, 207216.

Emotional Reactivity

Gray, J. A. (1982) The neuropsychology of anxiety Dawson, G. R. and Tricklebank M. D. (1995) Use of the elevated plus maze in the search for novel anxiolytic agents. TIPS 16, 33-36.

Morris Water Maze

Morris, R. G. M., Garrud, P., Rawlins, J. N. P. and O'Keefe, J. (1982) Place navigation impaired in rats with hippocampal lesions. Nature 297, 681-683.

Radial Arm Maze

Olton, D. S. and Samuelson, R. J. (1976) Remembrance-of places past: spatial memory in rats. Journal of Experimental Psychology: Animal Behaviour Processes 2, 97-116.

T Maze

Rawlins, J. N. P. and Olton, D. S. (1982) The septo-hippocampal system and cognitive mapping; Behavioural Brain Research 5, 331-358.

The examples below further illustrate the invention.

### EXAMPLES

### Example 1

Direct Injection of AChE Peptide into Rat Hippocampus—Histological Effects

AChE peptide was injected into rat hippocampus and subsequently the brains were examined under a light microscope. The hippocampus was-chosen as the most suitable brain structure in which to show effects of AChE peptide as it is very vulnerable to neurotoxins and damage from other causes such as ischaemia. Post mortem histological. examination or pre mortem brain scans reveal prominent decay of the hippocampus in Alzheimer brains.

Detailed Method

Male Wistar or Dark Agouti (DA) rats were anaesthetised with Avertin and injected with 0.5 μl of 2 mM AChE peptide (DA rats received 1 μl of 1 mM) in the right hippocampus. An equivalent control injection was made in the left hippocampus. After recovery from the anaesthetic and a few days in their home cages rats were deeply anaesthetised and transcardially perfused with formalin preservative, the brains removed and prepared for microscopical examination. Sections of 50 μm were cut on a sliding microtome and stained with cresyl violet to show the nerve cells. The amount of neural damage was estimated by counting the length of hippocampal cell layers destroyed (quantified in terms of microscope graticule units).

Results

In the most symmetrically injected DA rat (to give the fairest comparison between control and AChE peptide injected sides of the hippocampus) there were 25 units of damage on the control side, 119 on the AChE peptide injected side.

In a systematic assessment of the Wistar rat brains, there was a mean of 7.4 units of damage on the control side, 155.3 on the AChE peptide side. The difference between control and AChE peptide sides was statistically significant (P=0.008).

NB. In the above example, a P value of 0.008 means the probability of this result occurring by pure chance is only 8 parts in 1,000.

These results show small areas of damage after injection of AChE peptide into the hippocampus, a crucial brain structure in memory and Alzheimer's disease.

Comparison with NMDA

A small number of injections were made into the hippocampus of DA rats with the reference neurotoxin NMDA (N-methyl D-aspartic acid) at an equivalent concentration to that of AChE peptide used above (1 μl of 1 mM). Histological examination of these brain sections showed small areas of neural damage.

Since the concentration of NMDA used to produce experimental neural destruction (as a tool in experimental psychology to examine the function of a particular brain region) is much higher (typically 68 mM) a small number of rats were injected with this larger amount, in a volume of 1 μl, in comparison with an equivalent amount of AChE peptide. Results showed that AChE peptide produced local hippocampal damage, whereas that from NMDA was more widespread.

One of the two rats given AChE peptide at this higher dose looked very sick 5 days after the surgery and was therefore deeply anaesthetised and the brain prepared for microscopy. Severe body weight loss was also noted. The other rat survived 11 days until it was finally anaesthetised, but it too lost some weight. This supported informal observations from the earlier, low dose experiment that AChE peptide treated rats might lose more weight than is normal after such surgery.

The brain of this surviving rat was prepared for microscopical examination as above. There was a small amount of (presumably mechanical) damage on the left (control) injection site along the line of the injection needle. The right side, injected with AChE peptide, showed much more extensive damage, particularly in the overlying cortex, probably due to AChE peptide solution refluxing up the track left by the injector as it was withdrawn. The hippocampus itself showed a small localised area of cell loss. This appeared less than after injection of the reference neurotoxin NMDA, which typically extended over many consecutive brain sections.

### Example 2

Effects of AChE Peptide Injected into the Medial Septum/Diagonal Band of Broca (S/DB) on Behaviour, Weight and Histology

Methods

Wistar rats (175-200 g) were received from the suppliers (Harlan UK) and acclimated to the laboratory for three weeks before being trained on a delayed non-matching to sample (DNMS) task on an elevated wooden T-maze. In this task they are mildly food deprived and receive food pellets with enhanced flavour and nutritional content as rewards.

The DNMS task capitalises on the rat's innate tendency to alternate which cross arm of the T-maze it runs along; this derives from its normal foraging behaviour, where returning to a place from which it has recently eaten all the food is unlikely to pay dividends.

Each rat is placed at the start of the stem of the T-maze and allowed to run to retrieve a food pellet at the end of one cross arm. Access to the other cross arm is blocked. This block is then removed and the rat returned to the start. Normal rats choose to go to the opposite arm to that recently visited on this free choice part of the trial. After being allowed to consume their reward (if a correct choice was made) they are returned to their home cage and their cagemate is then tested in the same way. Typically a squad of ten rats is run in a “round robin’ fashion, so up to fifteen minutes intervenes between one trial and the next. Normal rats excel at this task once initial training (chiefly to familiarise them with the new smells on the maze and the elevated position off the floor) is complete.

The rats in the present experiment were given forty trials on the T-maze, then divided into groups matched for performance (all were above 90% correct). They were returned to unlimited food for a few days before undergoing surgery under Avertin anaesthesia.

The experimental group (n=6 rats) received an injection of AChE peptide into the medial septum/vertical limb of the diagonal band (S/DB). Control rats (n=6 rats) received an equal amount of the vehicle, water, in the same way, while another group (n=5 rats) received an injection of the reference neurotoxin NMDA at an equivalent dose to that of AChE peptide (2 μl of 33 mM). Each injection was given slowly over 15 minutes through a 34 gauge stainless steel injection needle coupled by polyethylene tubing to a 10 μl Hamilton syringe. Co-ordinates used on the stereotaxic instrument (Kopf) were 0.7 mm anterior to bregma, 1.0 mm lateral to midline and 6.5 mm from skull surface at bregma. The incisor bar was set at −0.5 mm for all rats, resulting in approximately a level skull surface between the bregma and lambda sutures. The arm of the stereotaxic instrument was angled at 10 degrees to vertical to avoid damage to the sagittal sinus.

Post-operatively the rats were weighed every day. Approximately two weeks after operation they were again food restricted and tested for performance on the T-maze.

After forty trials conducted exactly as before their brain operation, the task was made more difficult by giving twenty massed trials (i.e. without the normal 10-15 minute gap between each individual trial). A further more difficult variant was to impose an approximately 45 second delay between sample and choice trials. The rat would be returned to its home cage and partner during the delay, and this would serve as a “forget” cue as it normally signalled the end of one trial and a waiting period before beginning the next one.

After completion of T-maze testing rats were returned to a free feeding regimen before being tested on two successive days in standard locomotor activity cages. Each cage was equipped with two infrared beams to detect movement.

After a final weighing, rats were killed by stunning followed by decapitation. The hippocampus and cingulate/secondary motor cortex were rapidly removed and stored frozen at −80 degrees Celsius. The block of brain anterior to the hippocampus was placed in 30% sucrose formalin and left to fix for at least a week prior to sectioning on a microtome and staining with cresyl violet. Microscopical brain examination was subsequently performed as in earlier experiments.

Results

Weight and Behaviour

All rats lost some weight after surgery. Whereas NMDA treated rats lost more weight than controls, they regained it quicker than the AChE peptide treated rats which were statistically significantly slower than either controls or NMDA rats to recover their weight (see FIG. 2).

It was noted that one AChE peptide rat in particular seemed to display aberrant behaviour typical of hippocampally lesioned rats immediately post-operatively; it was, hyperdefensive, displaying an “upright boxing” posture to both partner and experimenter.

When tested post-operatively on the T-maze, AChE peptide treated rats showed twice the deficit seen in the NMDA group on the first block of ten trials. The latter's performance subsequently returned to control levels, whereas that of the AChE peptide treated group remained low (see FIG. 3).

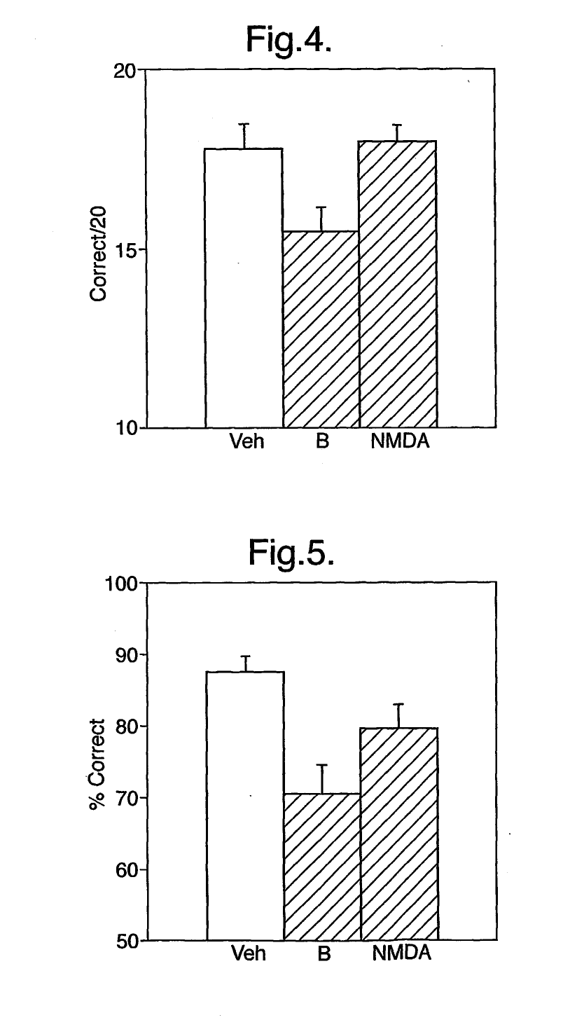

Massing the trials had little effect on the performance of the control and NMDA groups, while that of the AChE peptide treated group continued to be poor (see FIG. 4).

Imposing a delay between sample and choice produced a non-significant decrease in the NMDA group relative to control, whereas the deficit in the AChE peptide group was significantly greater than both (see FIG. 5).

The ANOVA (Analysis of Variance) statistical test performed on the locomotor activity test data showed that there were significant effects of days (i.e. activity was less on day 2 than day 1) and group (the AChE peptide group B was more active than the controls, which were not significantly different from NMDA) (see FIG. 6).

The final weighing (see FIG. 7) showed that all rats eventually gained weight before being anaesthetised approximately one month after the operation.

Histology

There were only small effects of NMDA on the histological appearance of the septal area. In the AChE peptide treated group, however, and especially in the two rats which were the worst performers on the T-maze, there was striking damage to not only the septal region itself but also the fornix which is the axonal pathway from the septum to the hippocampus (see FIG. 9). In the worst cases these structures had virtually disappeared. This was totally unexpected given the localised damage seen after the earlier experiment with hippocampal injections, and was unlike anything normally seen after conventional neurochemical lesions.

Further experiments and replications in the same (Wistar) strain of rats and a different one (Dark Agouti) have shown that the behavioural and histological effects of the AChE peptide are replicable.

Use of the AChE Peptide to Model Alzheimer's Disease.

These results suggest a number of ways that the AChE peptide could be used in the search for therapies for Alzheimer's disease. Typically it could be co-administered with a putative therapeutic agent and the behaviour and brains of the animals could be examined to see if the characteristic AChE peptide dysfunctions were prevented. Animal species other than rodents such as primates could be used for different or more sophisticated paradigms.

### Example 3

Experiments to Compare Effects of AChE Peptide Against Butyrylcholinesterase Peptide (Peptide C) and a Scrambled Version of AChE Peptide

Methods

Wistar rats were anaesthetised and injected as described in Example 2, with one of the following. AChE peptide, the equivalent peptide from BuChE, a scrambled version of the AChE peptide: HSWRAEVFHKYWSM, NMDA and water as a control. The number of rats in each group was between four and five.

Brain examination was performed as described in Example 2.

Results

Significant tissue loss was observed in the AChE peptide treated rats (see FIG. 9).

### Example 4

Effect of Biotinylated AChE Peptide Injected into the Nucleus Basalis of Meynert of Rafts on Performance in a Serial Choice Reaction Task

Test for Cognitive Function

Testing for loss of cognitive function reminiscent of loss of such function associated with Alzheimer's Disease in humans was carried out using a serial choice reaction task, a preferred form of task for testing for attentional deficits, as described by Higgs et al. (Higgs, S., Deacon, R. M. J. & Rawlins, J. N. P., European Journal of Neuroscience (2000)12, 1781-1788).

Apparatus

A rat testing cubicle with a retractable lever on the front wall was employed as illustrated in FIG. 11. The back wall was concave with three food magazines, one in each corner and one in the centre. A light was present within each corner magazine. Magazine entries were recorded by microswitches attached to panels covering the entrances. The entire apparatus was computer-controlled.

Procedure

Rats are trained to press the lever when it is presented. This results in one of three events: a light can flash either from the left magazine, or the right magazine, or there is no light. The rat will find a reward (a 45 mg food pellet) respectively in the left, right or centre magazine tray. Thus, a left response to a right flash (incorrect) suggests that the rat saw the flash but was not paying sufficient attention to where it came from. A response to the centre tray would suggest that he thought there had been no light flash.

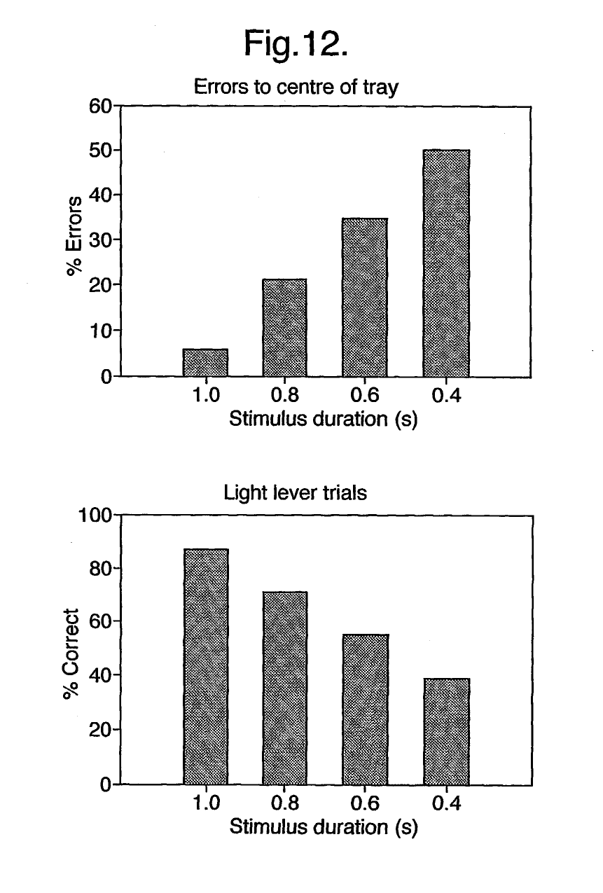

With control rats, as the light stimulus duration is decreased from 1.0 s to 0.4 s, response accuracy has been found to decrease. This is reflected as an increase of errors to the centre tray, as if the rats had not seen a light (Higgs et al., ibid, Experiment 3; see also appended FIG. 12)

Animal Treatment

A cohort of rats were trained on the attentional task (light stimulus duration 1.0 s). When performance was stable, they were divided into three matched groups, each with seven or more rats. Under deep anaesthesia, each rat was placed in a stereotaxic frame, with the incisor bar set at −3 mm to give a level head. Bilateral-injections, each of 2 μl, were made into the NBM area (see FIG. 13). The control group of rats received injections of water. The second group of rats received an aqueous solution of biotinylated AChE peptide and the third group received a solution of the known neurotoxin NMDA. The concentration of biotinylated AChE peptide and NMDA employed was 16.5 mM. Rats were allowed at least a week to recover from the operation.

After a period of post-operative testing, the rats were terminally anaesthetised and perfused intracardially with ice cold saline. The brain was removed and a portion of cortex (including the frontal, parietal and temporal areas, but excluding visual and cingulate cortex) was removed from each hemisphere and frozen. The remaining brain was preserved in formalin and subsequently coronally sectioned and stained with cresyl violet. The position and extent of the lesions was then determined by microscopical examination.

The frozen cortical tissue was used to determine the effectiveness of the NBM lesion. Using the method of Fonnum (J. Neurochem. (1975) 24, 407-409), the levels of the acetylcholine synthesising enzyme, choline acetyltransferase, (ChAT) were measured.

Since a proportion of this enzyme is due to the cholinergic innervation from the NBM, a decrease of ChAT would corroborate visualisation of a lesion at the site of injection in the NBM itself.

Results

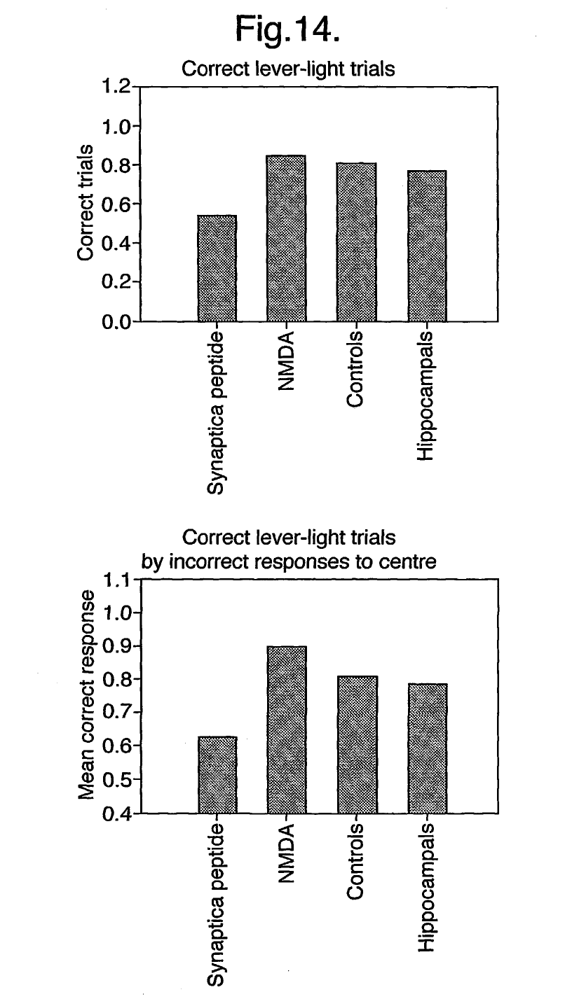

There was a selective effect of biotinylated AChE peptide on the attentional task. The proportion of correct lever-light trials was lower (FIG. 14, left panel) for rats treated with the biotinylated AChE peptide than for the controls. Thus, when a rat in the peptide-treated group pressed a lever and a light did come on, the rat frequently acted as if no light had been presented and defaulted to the centre tray (which would have been correct had there been no light (FIG. 14, right panel). Thus treatment with biotinylated AChE peptide produced a similar effect to decreasing light stimulus duration for control rats.

Microscopical examination revealed that the peptide injections had produced small or moderate size lesions in the NBM region.

The cholinergic loss in the cortex as measured by HPLC ChAT level was 12.1% for the NMDA comparison lesions, which didn't produce a significant reduction in correct lever-light trials, as compared to 8.4% for the peptide lesion.

Conclusion

Injection of biotinylated AChE peptide into rat NBM can produce histological damage which is functionally reflected in impairment in carrying out an attentional task. This effect on attention cannot be attributed to a selective cholinergic deficit since an equivalent injection of NMDA produces a greater reduction in cortical ChAT, but does not produce the same selective effect on attention. Injection of biotinylated AChE peptide, or an active variant thereof, into rat NBM is believed to represent a novel means of more accurately mirroring brain dysfunction in brains of Alzheimer's Disease patients, in particular attentional deficit.

Although the above discussed studies are confined to rats, it can be predicted that similar attentional deficit may be produced in other non-human animals by injecting biotinylated AchE peptide, or an effective variant thereof capable of causing comparable brain lesions, into the NBM.

1. A method of providing an animal model for a neurodegenerative disease which comprises introducing an effective amount of a peptide having the sequence:
AEFHRWSSYMVHWK (SEQ. ID. no. 1)
or an active variant of the peptide, into one or more sites in the brain of a non-human animal whereby said peptide causes cellular degeneration and thereby impairment of a testable brain function, wherein impairment of the same brain function in a human is indicative of a neurological disorder.

2. A method as claimed in claim 1 wherein said neurological disorder is Alzheimer's Disease and said impairment is impairment of a cognitive function.

3. A method as claimed in claim 2 wherein said impairment is an attentional deficit.

4. A method as claimed in claim 2 wherein the peptide is introduced into a site in the septohippocampal system.

5. A method as claimed in claim 4, wherein the peptide is introduced at the medial septum/diagonal band of Broca (S/DB) region of the brain.

6. A method as claimed in claim 2, wherein the peptide is introduced into a site in the cortical cholinergic system.

7. A method according to claim 6, wherein the peptide is introduced into the nucleus basalis magnocellularis (NBM).

8. A method according to claim 7 wherein the peptide of SEQ. ID. no. 1 is employed in biotinylated form or a variant thereof capable of providing functionally equivalent lesions in the NBM.

9. A method according to any one of claims 1 to 8 wherein said non-human animal is a rodent.

10. A method as claimed in any one of claims 1 to 9, which further comprises testing for said impairment.

11. An animal model for a neurodegenerative disease which is a non-human mammal treated with the peptide
AEFHRWSSYMVHWK (SEQ. ID. no 1)
or an active variant thereof in accordance with any one of claims 1 to 9.

12. An animal model according to claim 11 which is an animal model for Alzheimer's disease exhibiting attentional impairment.

13. An animal model according to claim 12 which is a rodent treated in accordance with claim 7 or claim 8.

14. A method as claimed in any one of claims 1 to 10 which further comprises administering prior to, simultaneously or after the peptide a test agent and determining whether said agent can inhibit, prevent or increase impairment of said testable brain function and/or can inhibit, prevent or increase cellular damage in the brain.

15. A method of testing an agent for biological activity in a neurodegenerative disorder which comprises administering said agent to an animal model prepared in accordance with any one of claims 1 to 10 and determining whether said agent will inhibit, prevent or increase impairment of said testable brain function and/or cause improvement or deterioration of cellular damage in the brain.

16. A method as claimed in claim 14 or claim 15 wherein said animal model is an animal model of Alzheimer's disease and said testable brain function is a cognitive function.

17. A method as claimed in claim 16 wherein impairment of attention is determined using apparatus providing a serial choice reaction task.

18. A method as claimed in claim 17 wherein said animal model is an animal model according to claim 13.

19. A test agent identified by a method according to any one of claims 14 to 18 which inhibits or prevents impairment of said testable brain function.

20. A method as claimed in any one of claims 14 to 18 wherein a test agent is selected which is a compound capable of inhibiting or preventing impairment of said testable brain function and which further comprises synthesising said compound.

21. A method as claimed in any one of claims 14, 15 and 20 which further comprises incorporating said agent into a pharmaceutical composition together with a pharmaceutically acceptable carrier or diluent.

22. A method as claimed in claim 21 wherein said agent is a compound and said pharmaceutical composition is suitable for delivery of said compound across the blood-brain barrier.

23. A pharmaceutical composition comprising a test agent as claimed in claim 19 together with a pharmaceutically acceptable carrier or diluent.

24. Use of an agent selected by a method as claimed in any one of claims 14 to 18 which inhibits or prevents impairment of said tested brain function for the manufacture of a medicament for use in the treatment of a neurological disorder.

25. Use of an agent selected by a method as claimed in any one of claims 16 to 18 which inhibits or prevents impairment of a tested cognitive function of relevance to Alzheimer's disease for use in the manufacture of a medicament for use in the treatment of said disease.
